(19) United States
(12) Patent Application Publication
(10) Pub. No.: US 2024/0321397 A1
(43) Pub. Date: Sep. 26, 2024

(54) EXTRACTING PROPERTIES FROM A SPARSE DATA SET BY APPLYING HYPERDIMENSIONAL COMPUTING AND DIMENSION REDUCTION

(71) Applicant: Accenture Global Solutions Limited, Dublin (IE)

(72) Inventor: Maziyar Baran Pouyan, Petaluma, CA (US)

(21) Appl. No.: 18/612,240

(22) Filed: Mar. 21, 2024

Related U.S. Application Data
(60) Provisional application No. 63/492,002, filed on Mar. 24, 2023.

(51) Int. Cl. G16B 30/00; G16B 40/20; G16H 10/40

(52) U.S. Cl. CPC G16B 30/00; G16B 40/20; G16H 10/40

(57) ABSTRACT

The present disclosure relates to a system, computer readable medium, and method for applying hyperdimensional computing and dimension reduction to extract properties from a sparse data set. Applying hyperdimensional computing can solve issues of dimensionality and dropout causing sparse data by expanding the dimension of the data. The result of hyperdimensional computing can involve too much data to be reasonably suitable for downstream computing processes (e.g., clustering for classification). Transforming the hyperdimensional embeddings provided by hyperdimensional computing into simplified/reduced embeddings can solve the problems of processing extremely large data. This improvement in accuracy and usefulness/useability of the sparse data helps reduce the need for extensive time, computing resources, and expensive equipment to extract expression data from deeper from cells.

### CROSS-REFERENCE TO RELATED APPLICATION

This application is a continuation-in-part of and claims the benefit of U.S. Provisional Patent Application Ser. No. 63/492,002 filed on Mar. 24, 2023, and titled “Extracting Properties from a Sparse Data Set by Applying Hyperdimensional Computing and Dimension Reduction”, the disclosure of which is incorporated by reference herein in its entirety.

### TECHNICAL FIELD

The present disclosure generally relates to extracting properties from a sparse data set. More specifically, the present disclosure relates to systems and methods for applying hyperdimensional computing and dimension reduction to extract properties from a sparse data set.

### BACKGROUND

Traditionally, ribonucleic acid (RNA) has been analyzed by bulk sequencing, which involves analyzing the genome of a cell population, such as a cell culture, a tissue, an organ, or entire organism, rather than individual cells. Bulk sequencing produces an average genome. Single cell sequencing produces genomes of individual cells that form a cell population. The advancement of single cell sequencing improves the ability to identify more granular properties of individual cells and to measure the RNA expression of a considerable amount of single cells simultaneously, resulting in noticeable progress in the knowledge of cellular structure. However, the advancement of single cell sequencing is an expensive endeavor. Extracting expression data much deeper from cells and the increased number of features/categories of expression data extracted requires extensive time and computational resources. For example, in many cases the number of columns in these kinds of data sets is in the range of 10,000 to 25,000. This data is extremely high in dimension and requires extensive time and computational resources to accurately capture the data for each column. For example, extracting accurate data on this level of detail and granularity from individual cells can take up to 100 million reads per cell.

When performed with less than ideal time and computational resources, extracting expression data from much deeper from cells and in such a large number of categories of properties can lead to sparse data (rather than robust data). In other words, many of these categories of properties of expression data end up empty or with a zero value (also known as dropout). Thus, the advancement of single cell sequencing presents a new problem of yielding sparse data sets. For example, in some instances, 90% of the values in a data table are zero.

There is a need in the art for a system and method that addresses the shortcomings discussed above.

### SUMMARY

A system and computer implemented method for applying hyperdimensional computing and dimension reduction to extract properties from a sparse data set is disclosed. In some embodiments, the method may include applying hyperdimensional computing to solve issues of dropout by expanding the dimension of the data. For example, in the field of single cell sequencing, single cell data can involve too much data to be reasonably suitable for downstream computing processes, such as clustering. And while the data is granular and can be too large for processing, the data may simultaneously be too sparse for processing. For example, downstream processes may include clustering, a critical step in single cell data analysis that can identify various cell populations and help researchers understand the cellular formation underlying cellular structures. However, clustering high-dimensional large-scale discrete yet sparse data, for example, by k-means clustering, achieves poor results. Sparse data lacks the details needed to distinguish data points (e.g., cells) to cluster the data points into groups. Many clustering techniques are not scalable for tens of thousands of cells. The disclosed techniques of hyperdimensional computing can expand the dimension of the data to fill in the gaps of the raw data. In other words, hyperdimensional computing can deal with incomplete, noisy, and ambiguous data by leveraging the properties of high-dimensional spaces. In such spaces, even if some aspects of data are missing or uncertain, the overall patterns can still be detected and used for further processing. However, once the raw data is expanded by hyperdimensional computing, the large raw data that was insufficiently large for downstream processes becomes even larger data with more dimensions.

To solve the problems of processing extremely large data produced by hyperdimensional computing, the disclosed system and method also include transforming the hyperdimensional embeddings provided by hyperdimensional computing into simplified/reduced embeddings that are more suitable for downstream computing processes while still retaining the meaningfulness of the data. In other words, the disclosed systems and methods enable the extremely granular data produced by hyperdimensional computing to be more quickly processed by various downstream computing processes with more accuracy and robustness.

The improvement in accuracy and usefulness/useability of the sparse data that is provided by the disclosed hyperdimensional computing and dimension reduction techniques help reduce the need for extensive time, computing resources, and expensive equipment to extract expression data from deeper from cells. In other words, rather than spending more time and resources (both computing and financial) on extensive readings, the raw, sparse data gathered with limited time and resources can be transformed into more usable data.

While the disclosed embodiments are discussed with the application of analyzing single cells, including RNA of cells, it is understood the disclosed embodiments can also be used with other applications. For example, the disclosed systems and methods can be used in other types of analysis involving extensive dimensionality, such as ecological, financial, actuarial, and healthcare applications. In some embodiments, the disclosed systems and methods include downstream computing processes applied to the embeddings resulting from hyperdimensional encoding and dimension reduction.

In one aspect, the disclosure provides a computer implemented method for applying hyperdimensional computing and dimension reduction to extract properties from a sparse data set. The method may include receiving initial data. The method may further include applying hyperdimensional encoding to the initial data to generate hyperdimensional representations. The method may further include applying feature construction from hyperdimensional space to the generated hyperdimensional representations to produce results. The method may further include performing a downstream process to the results of the feature construction. The method may further include presenting to a user via a display of a user interface the results of the downstream process.

The method may include collecting a human tissue sample. The method may include isolating a single cell from the human tissue sample. The method may include extracting initial data from the single cell. In some embodiments, extracting initial data from a single cell includes performing single cell ribonucleic acid sequencing (scRNA-seq) on the single cell to generate first scRNA-seq data, wherein the wherein the initial data includes the first scRNA-seq data. In other words, the method may include extracting genetic material from the single cell for analysis. The data (called single cell data) produced by this process can include gene expression values of thousands of cells in the sampled tissue. In other words, the single cell data is the gene expression values representing the genetic material. In some embodiments, the single cell data is the initial data that undergoes hyperdimensional encoding. In some embodiments, applying hyperdimensional encoding to the initial data to generate hyperdimensional representations includes encoding each gene of the single cell as a hypervector with D dimensions. In some embodiments, applying hyperdimensional encoding to the initial data to generate hyperdimensional representations includes performing randomized Singular Value Decomposition (SVD), including retrieving the first q Eigen values and vectors from the decomposition presented in the following equation:

$$C = {\overline{X}}^{sT} \times {\overline{X}}^{S} = V \times \sum \times {U}^{T} \times \sum \times {V}^{T} = V \times {\sum }^{2} \times {V}^{T} .$$

In some embodiments, feature construction includes feature construction from a cell-cell Pearson correlation of X_{n×D}^{s}. In some embodiments, the downstream process is clustering. In some embodiments, the downstream process is trajectory detection. The features in this paragraph may also apply to the embodiments of systems and non-transitory medium described below.

In yet another aspect, the disclosure provides a system for applying hyperdimensional computing and dimension reduction to extract properties from a sparse data set, comprising one or more computers and one or more storage devices storing instructions that are operable, when executed by the one or more computers, to cause the one or more computers to perform the following: (1) receiving initial data; (2) applying hyperdimensional encoding to the initial data to generate hyperdimensional representations; (3) applying feature construction from hyperdimensional space to the generated hyperdimensional representations to produce results; (4) performing a downstream process to the results of the feature construction; and (5) presenting to a user via a display of a user interface the results of the downstream process.

In yet another aspect, the disclosure provides a non-transitory computer-readable medium storing software comprising instructions executable by one or more computers which, upon such execution, cause the one or more computers to apply hyperdimensional computing and dimension reduction to extract properties from a sparse data set by: (1) receiving initial data; (2) applying hyperdimensional encoding to the initial data to generate hyperdimensional representations; (3) applying feature construction from hyperdimensional space to the generated hyperdimensional representations to produce results; (4) performing a downstream process to the results of the feature construction; and (5) presenting to a user via a display of a user interface the results of the downstream process.

Other systems, methods, features, and advantages of the disclosure will be, or will become, apparent to one of ordinary skill in the art upon examination of the following figures and detailed description. It is intended that all such additional systems, methods, features, and advantages be included within this description and this summary, be within the scope of the disclosure, and be protected by the following claims.

While various embodiments are described, the description is intended to be exemplary, rather than limiting, and it will be apparent to those of ordinary skill in the art that many more embodiments and implementations are possible that are within the scope of the embodiments. Although many possible combinations of features are shown in the accompanying figures and discussed in this detailed description, many other combinations of the disclosed features are possible. Any feature or element of any embodiment may be used in combination with or substituted for any other feature or element in any other embodiment unless specifically restricted.

This disclosure includes and contemplates combinations with features and elements known to the average artisan in the art. The embodiments, features, and elements that have been disclosed may also be combined with any conventional features or elements to form a distinct invention as defined by the claims. Any feature or element of any embodiment may also be combined with features or elements from other inventions to form another distinct invention as defined by the claims. Therefore, it will be understood that any of the features shown and/or discussed in the present disclosure may be implemented singularly or in any suitable combination. Accordingly, the embodiments are not to be restricted except in light of the attached claims and their equivalents. Also, various modifications and changes may be made within the scope of the attached claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, reference numerals designate corresponding parts throughout the different views.

FIG. 1 is a schematic diagram of a system for applying hyperdimensional computing and dimension reduction to extract properties from a sparse data set, according to an embodiment.

FIG. 2 is a schematic diagram of an overview of methods of extracting gene data.

FIG. 3 is a schematic diagram of an overview of a single cell multi-omics and further downstream processes, according to an embodiment.

FIG. 4 is a schematic diagram of an overview of a method for applying hyperdimensional computing, according to an embodiment.

FIG. 5 is a schematic diagram of an overview of hyperdimensional computing, according to an embodiment.

FIG. 6 is a schematic diagram of an overview of dimension reduction, according to an embodiment.

FIG. 7 is a flowchart of a method for applying hyperdimensional computing, according to an embodiment.

### DESCRIPTION OF EMBODIMENTS

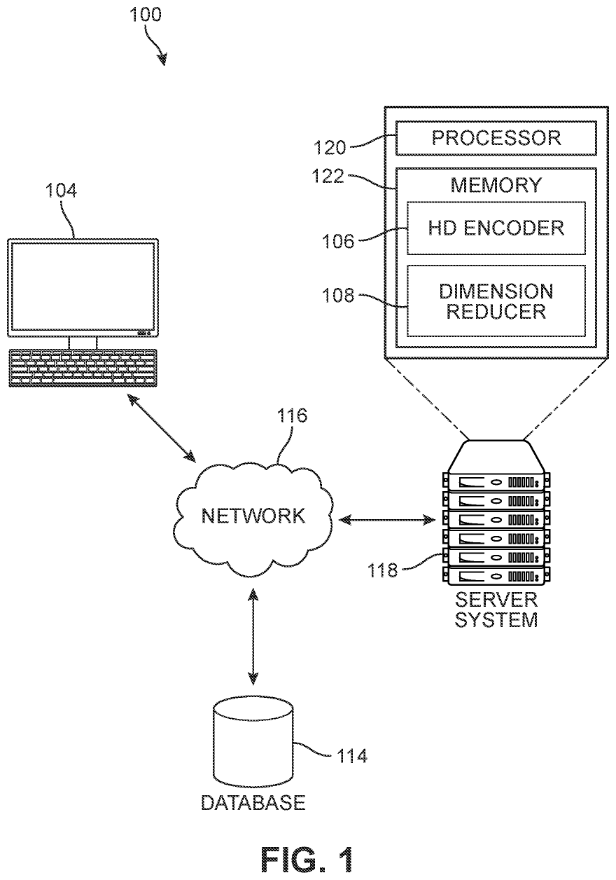

FIG. 1 is a schematic diagram of a system for applying hyperdimensional computing and dimension reduction to extract properties from a sparse data set 100 (or system 100), according to an embodiment. System 100 may include a user and user device 102 (or device 102 or user 102). During use, a user may interact with the system to create hyperdimensional embeddings and to reduce the dimension of the hyperdimensional embeddings to generate more useable embeddings.

The disclosed system may include a plurality of components capable of performing the disclosed computer implemented method. For example, system 100 includes a user device 104, a computing system 118, and a database 114. Database 114 may store information that is to be processed and transformed by the disclosed method. For example, in embodiments involving single cell sequencing, database 114 may store databases containing information about one or more cells. In another example, database 114 may store arrays or lists of information about one or more cells.

The components of system 100 can communicate with each other through a communication network 116. For example, user device 104 may retrieve information about a cell from database 114 via communication network 116. In some embodiments, communication network 116 may be a wide area network (“WAN”), e.g., the Internet. In other embodiments, communication network 116 may be a local area network (“LAN”).

While FIG. 1 shows one user device, it is understood that one or more user devices may be used. For example, in some embodiments, the system may include two or three user devices. In some embodiments, the user devices may be computing devices used by a user. For example, user device 102 may include a smartphone or a tablet computer. In other examples, user device 102 may include a laptop computer, a desktop computer, and/or another type of computing device. The user devices may be used for inputting, processing, and displaying information. In some embodiments, a digital camera may be used to generate images used for analysis in the disclosed method. In some embodiments, the user device may include a digital camera that is separate from the computing device. In other embodiments, the user device may include a digital camera that is integral with the computing device, such as a camera on a smartphone or tablet.

As shown in FIG. 1, in some embodiments, a preprocessor 104, a hyperdimensional encoder 106, and a dimension reducer 108 may be hosted in a computing system 118. Generally, hyperdimensional encoder 106 can embed raw input data (e.g., single cell gene expression) using a hyperdimensional computing technique and dimension reducer 108 can reduce the dimension of the embeddings created by hyperdimensional encoder 106 to generate embeddings that are more useable by downstream computing processes. Computing system 118 includes a processor 120 and a memory 122. Processor 120 may include a single device processor located on a single device, or it may include multiple device processors located on one or more physical devices. Memory 122 may include any type of storage, which may be physically located on one physical device, or on multiple physical devices. In some cases, computing system 118 may comprise one or more servers that are used to host the system.

Performance and efficiency of the disclosed hyperdimensional computing can be enhanced with specialized hardware or optimized data storage solutions. Additionally, given the high-dimensional vectors involved in hyperdimensional computing, in-memory computing can be advantageous. In-memory computing allows data to be processed directly in the system's RAM, reducing the need for frequent data transfers between storage and memory. In some embodiments, memory 122 can function as an “associative memory,” enabling the execution of in-memory computing.

The user may include an individual using the disclosed system to expand and reduce raw data, as well as perform downstream processes using the final reduced data. While FIG. 1 shows a single user device, it is understood that more user devices may be used. For example, in some embodiments, the system may include two or three user devices. The user device may be a computing device used by a user for communicating with the system. In some embodiments, one or more of the user devices may include a smartphone or a tablet computer. In other embodiments, one or more of the user devices may include a laptop computer, a desktop computer, and/or another type of computing device. The user devices may be used for inputting, processing, and displaying information. The user device may include a display that provides an interface for the user to input and/or view information.

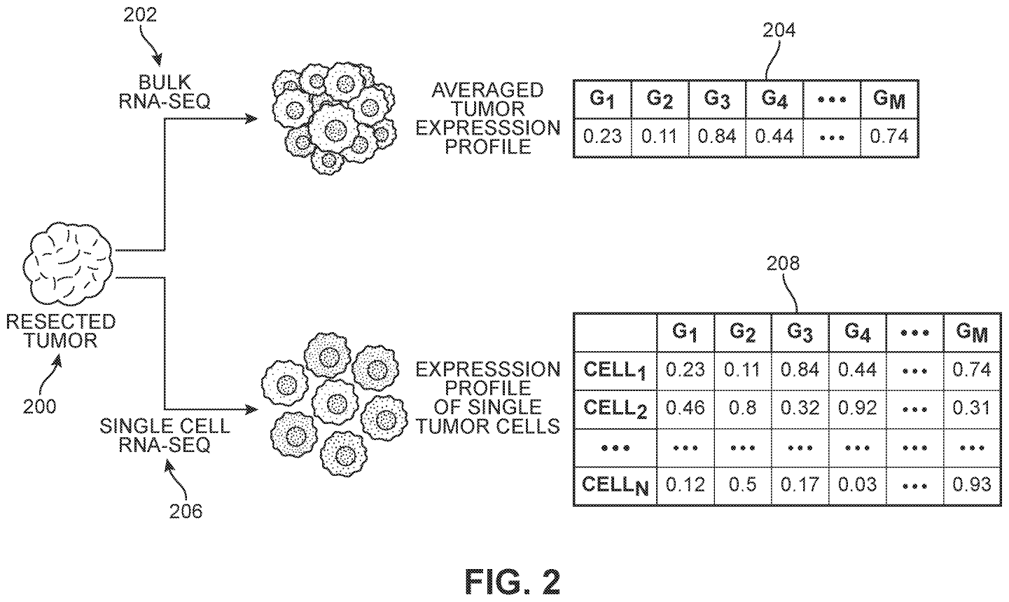

FIG. 2 is a schematic diagram of an overview of methods of extracting gene data to convey how single cell sequencing yields much more detailed information than bulk sequencing. In this example, a resected tumor sample 200 may undergo bulk RNA sequencing 202 to produce an averaged tumor expression profile. Table 204 shows an example of columns representing genes. The value for each column is an average gene expression value for all of the cells analyzed in resected tumor 200. Also in this example, the resected tumor sample may undergo single cell RNA sequencing 206 to produce an expression profile of single tumor cells. Table 208 shows an example of columns representing genes and rows representing individual cells from resected tumor sample 200. The value for each gene is specific to an individual cell. As discussed above, bulk sequencing produces an average genome, which is representative of broad strokes of a genome. Single cell sequencing produces genomes of individual cells that form a cell population. Table 204 next to table 208 demonstrates how much more information is extracted by single cell RNA sequencing than by bulk RNA sequencing. The advancement of single cell sequencing improves the ability to identify more granular properties of individual cells and to measure the RNA expression of a considerable amount of single cells simultaneously, resulting in greatly increasing the knowledge of cellular structure.

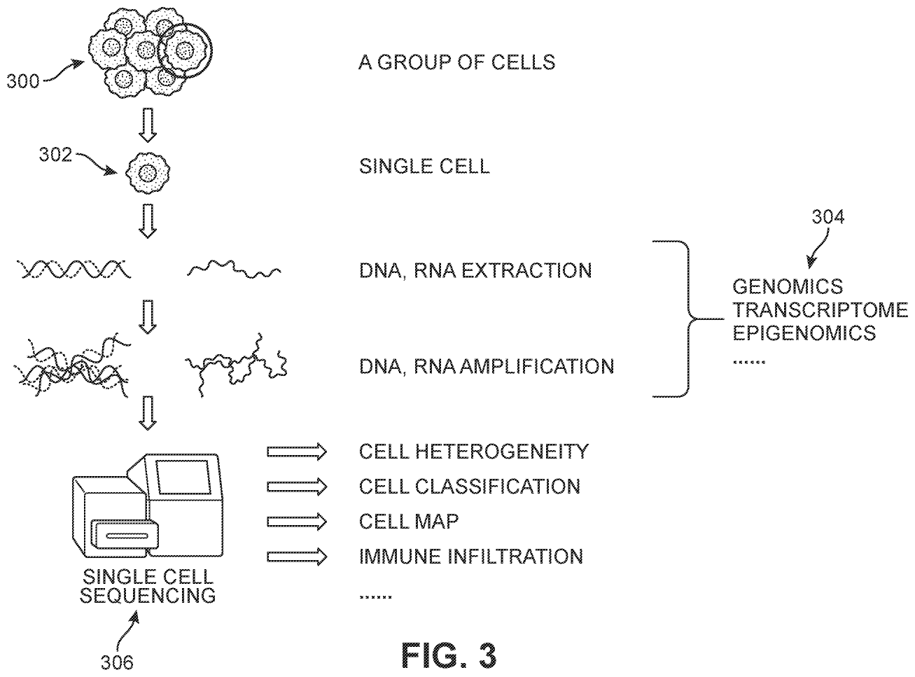

Embodiments may include single cell multi-omics and further downstream processes. FIG. 3 is a schematic diagram of an overview of a single cell multi-omics and further downstream processes, according to an embodiment. This example demonstrates how applying hyperdimensional computing and dimension reduction to extract properties from a sparse data set can be used in single cell multi-omics and further downstream processes. Single cell multi-omics can begin with single cell RNA sequencing, which can include collecting a group of cells 300, e.g., by resection. In some embodiments, the method may include collecting a human tissue sample. For example, a group of cells may be obtained from a human tissue sample. In some embodiments, the human tissue sample may be collected by resecting directly. In other embodiments, the human tissue sample may be collected by receiving an already-resected human tissue sample. Single cell RNA sequencing can further include isolating a single cell 302 from a cell population. The method may include isolating a single cell from the human tissue sample. In some embodiments, single cell RNA sequencing can include extracting, processing, and amplifying Deoxyribonucleic Acid (DNA) and RNA of each isolated cell to perform multi-omics 304, such as genomics, transcriptome, and epigenomics. The method may include performing single cell sequencing to generate data that can be used to perform downstream processes, such as determining cell heterogeneity, cell classification, generating a cell map, and identifying immune infiltration. The information generated by single cell sequencing can be sparse. The disclosed methods of hyperdimensional computing and dimension reduction can be applied to extract more granular, precise properties from the sparse data set.

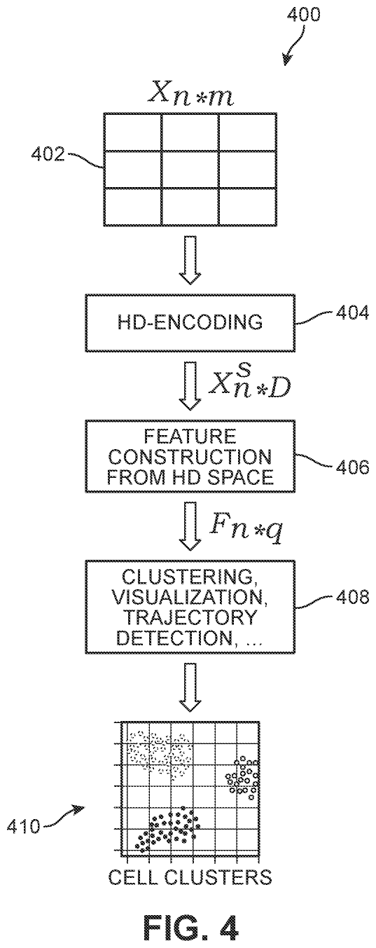

FIG. 4 is a schematic diagram of an overview of a method for applying hyperdimensional computing and dimension reduction to extract properties from a sparse data set 400 (or method 400), according to an embodiment. Method 400 may include receiving initial/raw data 402. For example, in the application of single cell analysis, this data may be from single cell gene expression data (e.g., with extensive categories of properties). The method may include applying hyperdimensional encoding 404 to initial/raw data 402 to generate hyperdimensional representations X_{n×D}^{s}. The method may further include applying feature construction 406 from hyperdimensional space to the generated hyperdimensional representations X_{n×D}^{s} to produce results F_{n×q}. The method may further include performing a downstream process 408, such as clustering, visualization, and/or trajectory detection, to the results F_{n×q} of the feature construction. FIG. 4 shows a result 410 of clustering, according to an embodiment.

Hyperdimensional encoding uses a fixed-size vector to represent a variable-length input, such as a sequence of symbols or a time series. The vector can be created by combining the input with a set of basis vectors, which can be chosen to be orthogonal and have many dimensions (e.g., a hypervector having thousands of dimensions). For example, hyperdimensional encoding may include mapping each data point to a high dimensional binary vector. This results in a high-dimensional representation that captures the statistical properties of the input. The ultra-wide data representation adds redundancy against noise. Hyperdimensional computing is robust, since information is dispersed evenly across each bit of the hypervectors. Hyperdimensional computing does not necessitate comprehensive understanding of the original data, as traditional learning algorithms do; instead, it employs a mapping function that encodes a given data into a high-dimensional space that simulates huge numbers of neurons and synapses in brains. So, due to the independence of dimensions in the Hyperdimensional-based learning models, they are particularly durable in the case of sparse and noisy data.

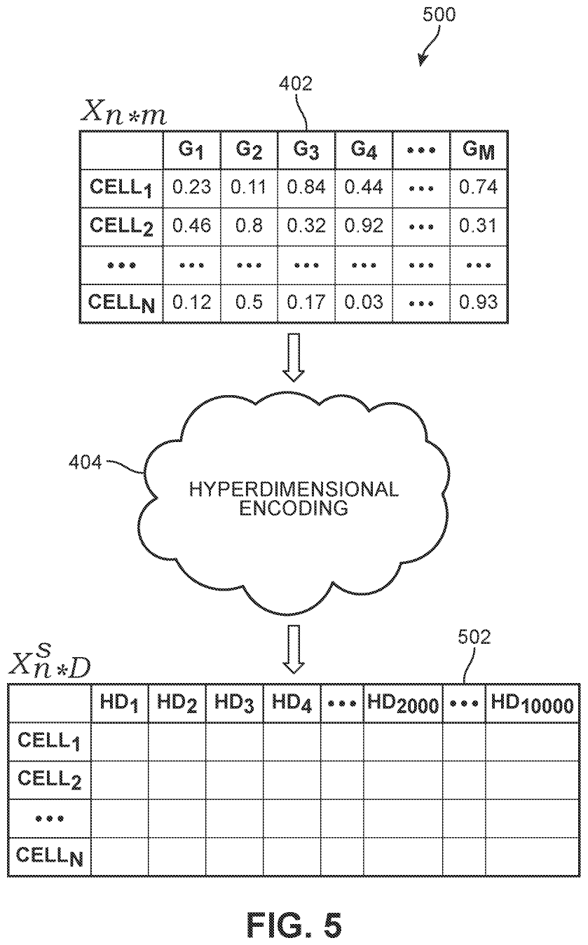

FIG. 5 is a schematic diagram of an overview of hyperdimensional computing, according to an embodiment. Hyperdimensional encoding 404 may be applied to initial/raw data 402 to generate hyperdimensional representations 502. In some embodiments, the method may include encoding each data point (e.g., cell) into a hypervector. In embodiments involving single cell data, the disclosed method may be performed to prepare single cell data for downstream processes. In such embodiments, the method may include gene filtering to prepare the single cell data for hyperdimensional encoding to ensure that the data undergoing hyperdimensional encoding is data of interest for the downstream processing. After applying initial filtering, the data can have m genes g=<g1, . . . , fm>(X_{n×m}). The goal is to encode each gene as a hypervector with D dimensions (e.g., D=10000). In the original domain, each gene vector can store the values across n cells.

To distinguish the position of each feature, the method may include randomly generating a set of base hypervectors, namely {B₁, B₂, . . . , Bₚ}, where p is the feature size of an original data point (Bᵢ∈{0,1}^{D}). Because of random generation and the size of the hypervector, the base hypervectors are nearly orthogonal, which means that the vectors only have about 50% of their elements in common by the following relationship:

$$δ ( {B}_{i} , {B}_{j} ) ≅ D / 2 ( 0 < i , j \leq m , i \neq j )$$

where δ denotes the similarity between the two hypervectors as measured by the Hamming distance.

The method may include employing a collection of level hypervectors to quantize the gene expression values. To do that, minimum and maximum gene values across cells, denoted by {gmin, gmax} may be computed. Then, these values may be discretized to Q levels linearly. The method may include producing a single hypervector representing first level, L1. The method may include randomly choosing D/Q bits and flipping them (i.e., converting 1 to 0 and 0 to 1 in the binary vectors). to form the next level hypervector (L2). The method may include continuing this process to generate LQ from LQ−1 vector and to generate a single hypervector for each level, {L1, L2, . . . , LQ}. This procedure assures that the last level hypervector, LQ, is approximately orthogonal to L1, while the others are correlated. For example, hypervectors assigned to neighbor levels have a strong correlation since they deviate by no more than D/Q bits.

The method may include aggregation. Aggregation may include encoding each cell by binding (XORing) of each base hypervector and the relevant level hypervector. Aggregation may include combining all of the results for all of the features as follows:

$${X}^{s} = \frac{B 1 ⊕ L 1 + B 2 ⊕ L 2 + … + Bm ⊕ Bm}{p}$$

where Lᵢ∈{L1, . . . , LQ}, and ⊕ represents an XOR operation and + is element − wise addition. X^{s} is the final hyperdimensional encoding version of the original data. All dimensions equally contribute to storing gene expression information during the aforementioned process to generate X^{s}. As a result, errors on data only affect a small portion of each hypervector and do not result in the loss of all information.

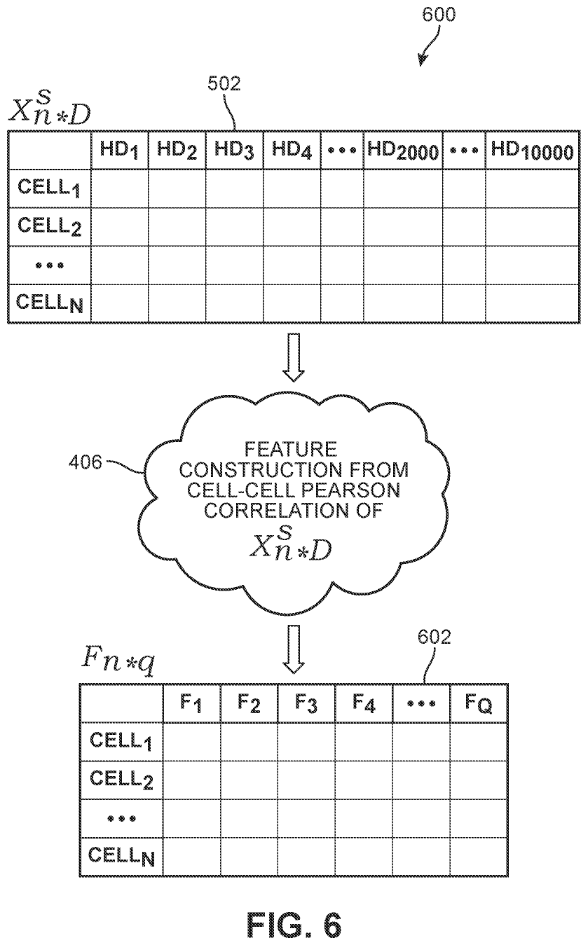

FIG. 6 is a schematic diagram of an overview of dimension reduction, according to an embodiment. The method may include applying feature construction 406 (or feature engineering 406) from hyperdimensional space to the hyperdimensional representations 502 generated by hyperdimensional encoding 404 to output results 602 of the feature construction. For example, in some embodiments, feature construction may include feature construction from cell-cell Pearson correlation of X_{n×D}^{s}.

In some embodiments, feature construction may include constructing features from X^{s} by applying random projection singular value decomposition (rSVD). It is noted that the matrix of cell-cell Pearson correlation coefficients can be written as:

$${C}_{n \times n} = {\overline{X}}_{n \times p}^{sT} \times {\overline{X}}_{p \times n}^{sT}$$

Where X^{S}∈ [symbol] ^{p×n} is the column-centered and column-scaled version of X, multiplied by a factor of 1/\sqrt{p−1}. Via the singular value decomposition of this matrix:

$${\overline{X}}_{p \times n}^{sT} = {U}_{p \times p} \times {\sum }_{p \times p} \times {V}_{p \times n}^{T}$$

The singular value/Eigen decomposition of the correlation matrix can be calculated as:

$$C = {\overline{X}}^{sT} \times {\overline{X}}^{S} = V \times \sum \times {U}^{T} \times \sum \times {V}^{T} = V \times {\sum }^{2} \times {V}^{T}$$

The low rank approximation of C∈ [symbol] ^{n×m} is available via singular value decomposition of X̅^{S}. The method may include performing randomized Singular Value Decomposition (SVD), including retrieving the first q Eigen values and vectors from the decomposition presented in the above equation. The method may include using these results as a feature matrix where cells are rows, and each cell is described by q features (columns). This feature matrix can be shown by F∈ [symbol] ^{n×q}. The following may be selected: q=[0.01n].

The results from feature construction may be used in performing downstream processes. For example, in various embodiments, downstream processes may include clustering, visualization (e.g., two-dimensional data visualization), and/or trajectory detection (which can provide inferences). Then, the method may result in output from downstream processes. For example, in one embodiment involving single cell analysis, the results of feature construction, which are smaller in dimension than the initial/raw data and the hyperdimensional representations, may be used for the downstream process of clustering. The output of clustering may include clusters of cells with similar properties/characteristics. The output may be presented to a user via a display of a user interface.

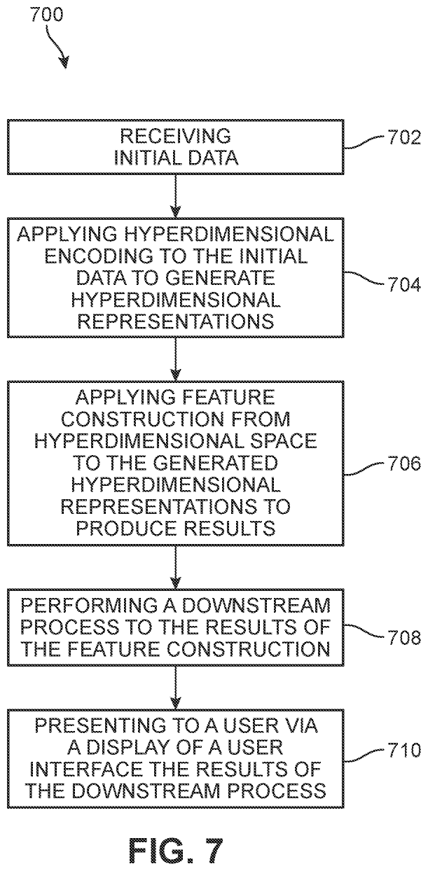

FIG. 7 shows a computer implemented method for applying hyperdimensional computing and dimension reduction to extract properties from a sparse data set 700 (or method 700), according to an embodiment. The method may include receiving initial data (operation 702). The method may further include applying hyperdimensional encoding to the initial data to generate hyperdimensional representations (operation 704). The method may further include applying feature construction from hyperdimensional space to the generated hyperdimensional representations to produce results (operation 706). The method may further include performing a downstream process to the results of the feature construction (operation 708). The method may further include presenting to a user via a display of a user interface the results of the downstream process (operation 710).

Embodiments may include a non-transitory computer-readable medium (CRM) storing software comprising instructions executable by one or more computers which, upon such execution, cause the one or more computers to perform the disclosed methods. Non-transitory CRM may refer to a CRM that stores data for short periods or in the presence of power such as a memory device or Random Access Memory (RAM). For example, a non-transitory computer-readable medium may include storage components, such as, a hard disk (e.g., a magnetic disk, an optical disk, a magneto-optic disk, and/or a solid state disk), a compact disc (CD), a digital versatile disc (DVD), a floppy disk, a cartridge, and/or a magnetic tape.

Embodiments may also include one or more computers and one or more storage devices storing instructions that are operable, when executed by the one or more computers, to cause the one or more computers to perform the disclosed methods.

Cloud computing environment can include, for example, an environment that hosts the policy management service. The cloud computing environment may provide computation, software, data access, storage, etc. services that do not require end-user knowledge of a physical location and configuration of system(s) and/or device(s) that hosts the policy management service. For example, a cloud computing environment may include a group of computing resources (referred to collectively as “computing resources” and individually as “computing resource”).

While various embodiments are described, the description is intended to be exemplary, rather than limiting and it will be apparent to those of ordinary skill in the art that many more embodiments and implementations are possible that are within the scope of the embodiments. Although many possible combinations of features are shown in the accompanying figures and discussed in this detailed description, many other combinations of the disclosed features are possible. Any feature or element of any embodiment may be used in combination with or substituted for any other feature or element in any other embodiment unless specifically restricted.

Other systems, methods, features, and advantages of the disclosure will be, or will become, apparent to one of ordinary skill in the art upon examination of the following figures and detailed description. It is intended that all such additional systems, methods, features, and advantages be included within this description and this summary, be within the scope of the disclosure, and be protected by the following claims.

This disclosure includes and contemplates combinations with features and elements known to the average artisan in the art. The embodiments, features, and elements that have been disclosed may also be combined with any conventional features or elements to form a distinct invention as defined by the claims. Any feature or element of any embodiment may also be combined with features or elements from other inventions to form another distinct invention as defined by the claims. Therefore, it will be understood that any of the features shown and/or discussed in the present disclosure may be implemented singularly or in any suitable combination. Accordingly, the embodiments are not to be restricted except in light of the attached claims and their equivalents. Also, various modifications and changes may be made within the scope of the attached claims.

We claim:

1. A computer implemented method for applying hyperdimensional computing and dimension reduction to extract properties from a sparse data set, comprising:
receiving initial data;
applying hyperdimensional encoding to the initial data to generate hyperdimensional representations;
applying feature construction from hyperdimensional space to the generated hyperdimensional representations to produce results;
performing a downstream process to the results of the feature construction; and
presenting to a user via a display of a user interface the results of the downstream process.

2. The computer implemented method of claim 1, further comprising:
collecting a human tissue sample;
isolating a single cell from the human tissue sample; and
extracting initial data from the single cell.

3. The computer implemented method of claim 2, wherein extracting initial data from a single cell includes performing single cell ribonucleic acid sequencing (scRNA-seq) on the single cell to generate first scRNA-seq data, wherein the wherein the initial data includes the first scRNA-seq data.

4. The computer implemented method of claim 2, wherein applying hyperdimensional encoding to the initial data to generate hyperdimensional representations includes encoding each gene of the single cell as a hypervector with D dimensions.

5. The computer implemented method of claim 2, wherein applying hyperdimensional encoding to the initial data to generate hyperdimensional representations includes performing randomized Singular Value Decomposition (SVD), including retrieving the first q Eigen values and vectors from the decomposition presented in the following equation:
$$C = {\overline{X}}^{sT} \times {\overline{X}}^{S} = V \times \sum \times {U}^{T} \times \sum \times {V}^{T} = V \times {\sum }^{2} \times {V}^{T} .$$

6. The computer implemented method of claim 1, wherein feature construction includes feature construction from a cell-cell Pearson correlation of X_{n×D}^{s}.

7. The computer implemented method of claim 1, wherein the downstream process is clustering.

8. The computer implemented method of claim 1, wherein the downstream process is trajectory detection.

9. A system for applying hyperdimensional computing and dimension reduction to extract properties from a sparse data set, comprising:
one or more computers and one or more storage devices storing instructions that are operable, when executed by the one or more computers, to cause the one or more computers to perform the following:
receiving initial data;
applying hyperdimensional encoding to the initial data to generate hyperdimensional representations;
applying feature construction from hyperdimensional space to the generated hyperdimensional representations to produce results;
performing a downstream process to the results of the feature construction; and
presenting to a user via a display of a user interface the results of the downstream process.

10. The system of claim 8, wherein extracting initial data from a single cell includes performing single cell ribonucleic acid sequencing (scRNA-seq) on the single cell to generate first scRNA-seq data, wherein the wherein the initial data includes the first scRNA-seq data.

11. The system of claim 9, wherein applying hyperdimensional encoding to the initial data to generate hyperdimensional representations includes encoding each gene of the single cell as a hypervector with D dimensions.

12. The system of claim 9, wherein applying hyperdimensional encoding to the initial data to generate hyperdimensional representations includes performing randomized Singular Value Decomposition (SVD), including retrieving the first q Eigen values and vectors from the decomposition presented in the following equation:
$$C = {\overline{X}}^{sT} \times {\overline{X}}^{S} = V \times \sum \times {U}^{T} \times \sum \times {V}^{T} = V \times {\sum }^{2} \times {V}^{T} .$$

13. The system of claim 8, wherein feature construction includes feature construction from a cell-cell Pearson correlation of X_{n×D}^{s}.

14. The system of claim 8, wherein the downstream process is clustering.

15. A non-transitory computer-readable medium storing software comprising instructions executable by one or more computers which, upon such execution, cause the one or more computers to apply hyperdimensional computing and dimension reduction to extract properties from a sparse data set by:
receiving initial data;
applying hyperdimensional encoding to the initial data to generate hyperdimensional representations;
applying feature construction from hyperdimensional space to the generated hyperdimensional representations to produce results;
performing a downstream process to the results of the feature construction; and
presenting to a user via a display of a user interface the results of the downstream process.

16. The non-transitory computer-readable medium of claim 15, wherein extracting initial data from a single cell includes performing single cell ribonucleic acid sequencing (scRNA-seq) on the single cell to generate first scRNA-seq data, wherein the wherein the initial data includes the first scRNA-seq data.

17. The non-transitory computer-readable medium of claim 16, wherein applying hyperdimensional encoding to the initial data to generate hyperdimensional representations includes encoding each gene of the single cell as a hypervector with D dimensions.

18. The non-transitory computer-readable medium of claim 16, wherein applying hyperdimensional encoding to the initial data to generate hyperdimensional representations includes performing randomized Singular Value Decomposition (SVD), including retrieving the first q Eigen values and vectors from the decomposition presented in the following equation:
$$C = {\overline{X}}^{sT} \times {\overline{X}}^{S} = V \times \sum \times {U}^{T} \times \sum \times {V}^{T} = V \times {\sum }^{2} \times {V}^{T} .$$

19. The system of claim 15, wherein feature construction includes feature construction from a cell-cell Pearson correlation of X_{n×D}^{s}.

20. The non-transitory computer-readable medium of claim 15, wherein the downstream process is clustering.
